(19) United States
(12) Patent Application Publication
(10) Pub. No.: US 2026/0309697 A1
(43) Pub. Date: Oct. 8, 2026

(54) BODY ODOR CARE SUPPORT INFORMATION PROVIDING DEVICE AND BODY ODOR CARE SUPPORT INFORMATION PROVIDING METHOD

(71) Applicants: AISIN CORPORATION, Kariya-shi (JP); TOKAI UNIVERSITY EDUCATIONAL SYSTEM, Tokyo (JP)

(72) Inventors: Hideaki YAMAGUCHI, Kariya-shi (JP); Eiji FUJIOKA, Kariya-shi (JP); Kanae MARUYAMA, Kariya-shi (JP); Yoshika SEKINE, Hiratsuka-shi (JP)

(73) Assignees: AISIN CORPORATION, Kariya-shi (JP); TOKAI UNIVERSITY EDUCATIONAL SYSTEM, Tokyo (JP)

(21) Appl. No.: 19/576,507

(22) Filed: Mar. 24, 2026

(30) Foreign Application Priority Data
Apr. 3, 2025 (JP) 2025-061879

(51) Int. Cl. G01N 33/497

(52) U.S. Cl. CPC G01N 33/497

(57) ABSTRACT

A body odor care support information providing device includes an acquisition unit, a calculation unit, a determination unit, a generation unit, and an output unit. The acquisition unit acquires emanation amount information regarding emanation amounts of a plurality of types of skin gases emanated from a skin of a subject. The calculation unit calculates, based on the emanation amount information, at least one index value indicating a ratio of an emanation amount of a specific skin gas selected from the plurality of types of skin gases. The determination unit determines, based on the at least one index value, a stress state of the subject, and determines, based on the determined stress state, body odor classification of the subject. The generation unit generates, based on the body odor classification of the subject, body odor care support information regarding the subject. The output unit outputs the body odor care support information.

### CROSS REFERENCE TO RELATED APPLICATIONS

This application is based on and claims priority under 35 U.S.C. § 119 to Japanese Patent Application No. 2025-061879, filed on Apr. 3, 2025, the entire content of which is incorporated herein by reference.

### TECHNICAL FIELD

An embodiment of the present disclosure relates to a body odor care support information providing device and a body odor care support information providing method.

### BACKGROUND DISCUSSION

It is known that stress odor emanates from skin with stress, and effective body odor care is required. In addition, there is a technique for detecting stress of a subject on the basis of gas emanated from the skin of the subject as a human (See JP 2019-198648 A, JP 2020-120629 A, and JP 2020-132621 A).

It is clear that when a human is in a stress state, gases of an unpleasant component emanate from the human. On the other hand, various gas components are regarded as the stress odor. In addition, skin gas is an emanated mixture of gases of various components. For this reason, there is a possibility that the conventional body odor care mainly dealing with the gas of the characteristic component is not sufficient as a countermeasure.

A need thus exists for a body odor care support information providing device and a body odor care support information providing method which are not susceptible to the drawback mentioned above.

### SUMMARY

A body odor care support information providing device according to an embodiment includes, as an example, an acquisition unit, a calculation unit, a determination unit, a generation unit, and an output unit. The acquisition unit acquires emanation amount information regarding emanation amounts of a plurality of types of skin gases emanated from a skin of a subject. The calculation unit calculates, based on the emanation amount information, at least one index value indicating a ratio of an emanation amount of a specific skin gas selected from the plurality of types of skin gases. The determination unit determines, based on the at least one index value, a stress state of the subject, and determines, based on the determined stress state, body odor classification of the subject.

The generation unit generates, based on the body odor classification of the subject, body odor care support information regarding the subject. The output unit outputs the body odor care support information.

A body odor care support information providing method using a body odor care support information providing device, according to an embodiment incudes:

acquiring emanation amount information regarding emanation amounts of a plurality of types of skin gases emanated from a skin of a subject;

calculating, based on the emanation amount information, at least one index value indicating a ratio of an emanation amount of a specific skin gas selected from the plurality of types of skin gases;

determining, based on the at least one index value, a stress state of the subject,

determining, based on the determined stress state, a body odor classification of the subject;

generating, based on the body odor classification of the subject, body odor care support information regarding the subject; and

outputting the body odor care support information.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a diagram illustrating an example of a hardware configuration of a body odor care support information providing device according to an embodiment;

FIG. 2 is a diagram illustrating an example of a detection position of skin gas according to the embodiment;

FIG. 3 is a diagram illustrating an example of a functional configuration of the body odor care support information providing device according to the embodiment;

FIG. 4 is a diagram illustrating an example of a mental and physical state two-dimensional map showing a relationship between a blood vessel index value, a metabolic index value, and a stress state;

FIG. 5 is a diagram illustrating another example of the mental and physical state two-dimensional map showing the relationship between the blood vessel index value, the metabolic index value, and the stress state;

FIG. 6 is a diagram illustrating an example of body odor care support information;

FIG. 7 is a diagram illustrating another example of the body odor care support information;

FIG. 8 is a flowchart illustrating an example of a flow of body odor care support information generation processing;

FIG. 9 is a diagram illustrating an example of a form of providing the body odor care support information to a subject;

FIG. 10 is a diagram illustrating an example of a relationship between the blood vessel index value and a stress value; and

FIG. 11 is a diagram illustrating an example of a relationship between the metabolic index value and a physical stress value.

### DETAILED DESCRIPTION

Hereinafter, a body odor care support information providing system and a body odor care support information providing device according to the present embodiment will be described with reference to the drawings. The configurations of the embodiment described below and the functions and effects brought by the configurations are merely examples, and the present disclosure is not limited to the following description.

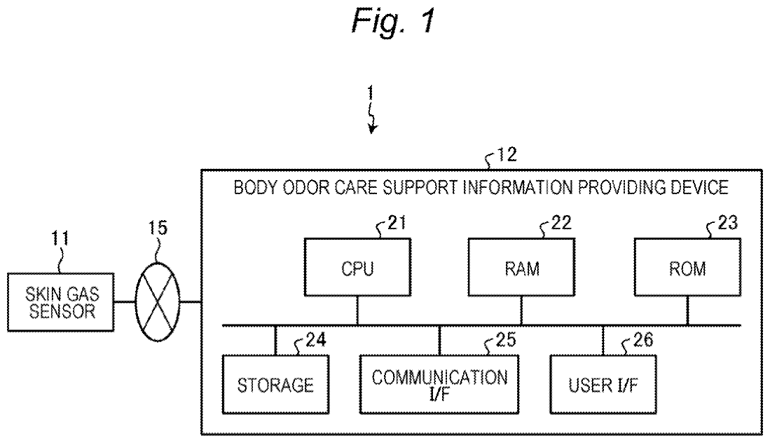

FIG. 1 is a diagram illustrating an example of a hardware configuration of a body odor care support information providing system 1 according to an embodiment. The body odor care support information providing system 1 according to the present embodiment includes a skin gas sensor 11 and a body odor care support information providing device 12.

The skin gas sensor 11 is a sensor that detects skin gas emanated from the skin of a subject. The skin gas sensor 11 is capable of detecting skin gas generated in association with activities for maintaining homeostasis in the human body, and is capable of detecting at least a blood-vessel-related gas related to vasomotion (contraction or expansion of blood vessels) and a metabolism-related gas related to metabolic activities.

The blood-vessel-related gas may be, for example, hexanoic acid, propionic acid, and the like.

The metabolism-related gas may be, for example, acetone, acetic acid, acetaldehyde, and the like.

The specific configuration of the skin gas sensor 11 is not particularly limited, but for example, an oxide semiconductor sensor, a quartz crystal microbalance (QCM) sensor, a field effect transistor (FET) sensor, a film type surface stress sensor, or the like may be used as the skin gas sensor 11. Further, skin gas emanation amount data obtained by quantitatively measuring skin gas collected by a passive flux sampler (PFS) for each skin gas type using a gas chromatography-mass spectrometry (GC-MS) device or the like can also be used as data of the skin gas sensor 11. The PFS is a small device that adsorbs and collects skin gas emanating from the skin surface of the subject on a solid trapping material, and thermally or chemically desorbs the skin gas from the trapping material to subject the desorbed skin gas to chemical analysis. An example of the PFS is a skin gas sampler (MonoTrap (registered trademark) SG DCC18) manufactured by GL Sciences Inc.

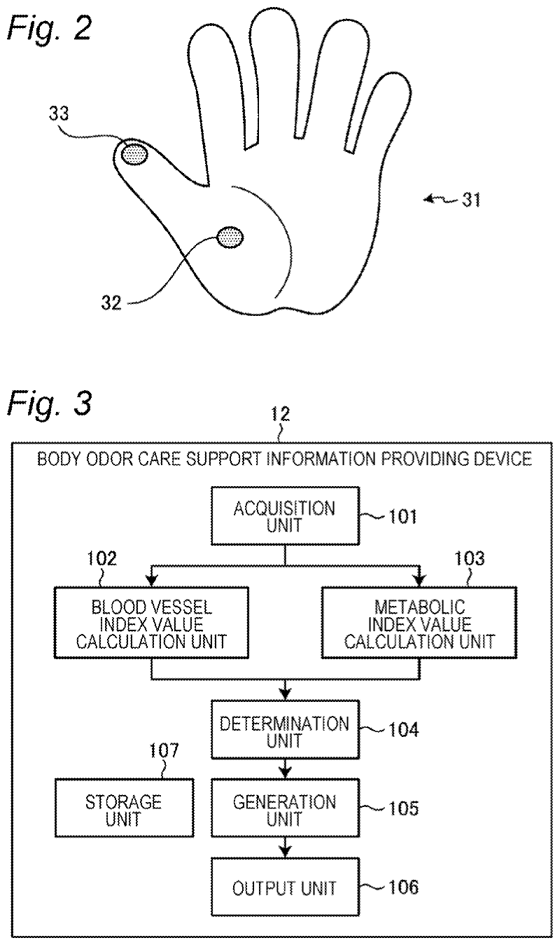

FIG. 2 is a diagram illustrating an example of a detection position of the skin gas according to the embodiment. The detection position of the skin gas by the skin gas sensor 11 according to the present embodiment is a palm portion 31 of the subject, and is particularly preferably a thenar eminence portion 32 or a fingertip portion 33. In these sites, since there are many capillaries, there is usually no hair, and there is little subcutaneous fat, the emanation amount of skin gas is large, and skin gas can be detected relatively stably. Note that, in FIG. 2, the fingertip of the thumb is exemplified as the fingertip portion 33, but the fingertip portion 33 may be the fingertip of another finger (index finger, middle finger, ring finger, or little finger).

Note that the detection position, specific configuration, installation position, and the like of the skin gas sensor 11 are not limited to the above. For example, the detection position may be a back-of-the-knee portion or the like.

The body odor care support information providing device 12 is connected to the skin gas sensor 11 via a predetermined network 15, and executes various types of information processing for detecting stress of the subject on the basis of a detection result of the skin gas sensor 11.

The body odor care support information providing device 12 exemplified herein is configured using a general-purpose information processing device (computer), and includes a central processing unit (CPU) 21, a random access memory (RAM) 22, a read only memory (ROM) 23, a storage 24, a communication interface (I/F) 25, and a user I/F 26.

The CPU 21 executes predetermined arithmetic processing using the RAM 22 as a work area according to a program stored in the ROM 23 or the storage 24. The storage 24 is a non-volatile memory such as a solid state drive (SSD) or a hard disk drive (HDD), and enables writing and reading of programs, data necessary for processing of the CPU 21, data generated by processing of the CPU 21, and the like. The communication I/F 25 is a device that enables communication with other electronic devices such as the skin gas sensor 11 connected via the predetermined network 15. The user I/F 26 is a device that enables acceptance of an input from a user (a user of the body odor care support information providing system 1 or the like), output of information to the user, and the like, and may be, for example, a keyboard, a pointing device, a touch panel mechanism, a display, a speaker, a microphone, or the like. The CPU 21, the RAM 22, the ROM 23, the storage 24, the communication I/F 25, and the user I/F 26 are connected via a data bus.

Note that the hardware configuration of the body odor care support information providing device 12 is not limited to the above, and may be configured using, for example, a field programmable gate array (FPGA), an application specific integrated circuit (ASIC), or the like, or may be a system in which a plurality of computers operate in cooperation.

FIG. 3 is a diagram illustrating an example of a functional configuration of the body odor care support information providing device 12 according to the embodiment. The body odor care support information providing device 12 according to the present embodiment includes an acquisition unit 101, a blood vessel index value calculation unit 102, a metabolic index value calculation unit 103, a determination unit 104, a generation unit 105, an output unit 106, and a storage unit 107. These functional units 101 to 107 are configured to function by cooperation of hardware elements and software elements (programs, firmware, or the like) constituting the body odor care support information providing device 12. In addition, at least some of these functional units 101 to 107 may be configured by dedicated hardware (a circuit or the like).

The acquisition unit 101 acquires, from the skin gas sensor 11, emanation amount information regarding an emanation amount of skin gas emanated from the skin of the subject. The acquisition unit 101 according to the present embodiment acquires blood-vessel-related gas information indicating the emanation amount of each of the plurality of types of blood-vessel-related gases and metabolism-related gas information indicating the emanation amount of each of the plurality of types of metabolism-related gases. The emanation amount may be, for example, a value indicating the amount of gas (detected by the skin gas sensor 11) emanated per predetermined unit time.

The blood-vessel-related gas information includes, for example, an emanation amount of hexanoic acid, an emanation amount of propionic acid, and the like.

The metabolism-related gas information includes, for example, an emanation amount of acetone, an emanation amount of acetic acid, an emanation amount of acetaldehyde, and the like.

Note that the emanation amount information acquired by the acquisition unit 101 may include information regarding an emanation amount of skin gas other than the blood-vessel-related gas and the metabolism-related gas, for example, other skin gas or the like emanated in accordance with homeostasis maintenance activities in the body.

The blood vessel index value calculation unit 102 calculates a blood vessel index value on the basis of the blood-vessel-related gas information acquired by the acquisition unit 101. The blood vessel index value is a value indicating a ratio of an emanation amount of a specific blood-vessel-related gas selected from a plurality of types of blood-vessel-related gases to a total emanation amount of the plurality of types of blood-vessel-related gases. For example, when the plurality of types of blood-vessel-related gases are hexanoic acid and propionic acid, the specific blood-vessel-related gas is hexanoic acid, an emanation amount of hexanoic acid is A1, and an emanation amount of propionic acid is A2, a blood vessel index value BV may be calculated by the following formula (1).

$$BV = A 1 / ( A 1 + A 2 ) \qquad ( 1 )$$

That is, the blood vessel index value BV can be a ratio of the emanation amount of hexanoic acid to the total emanation amount of hexanoic acid and propionic acid. Note that the above-described method for calculating the blood vessel index value BV is an example, and the selection of a plurality of types of blood-vessel-related gases and the selection of a specific blood-vessel-related gas are not limited to the above. For example, the blood vessel index value BV may be a ratio of the emanation amount of propionic acid to the total emanation amount of hexanoic acid and propionic acid (BV′=A2/(A1+A2)).

As described above, by using the blood vessel index value BV, which is the ratio of the emanation amount of the specific blood-vessel-related gas to the total emanation amount of the plurality of types of blood-vessel-related gases, it is possible to suppress the influence of the variation in the emanation amount of the skin gas caused by climatic conditions or factors unique to the subject (physical constitution, mental state, and the like).

The metabolic index value calculation unit 103 calculates a metabolic index value on the basis of the metabolism-related gas information acquired by the acquisition unit 101. The metabolic index value is a value indicating a ratio of an emanation amount of a specific metabolism-related gas selected from a plurality of metabolism-related gases to a total emanation amount of the plurality of types of metabolism-related gases. For example, when the plurality of types of metabolism-related gases are acetone, acetic acid, and acetaldehyde, the specific metabolism-related gas is acetone, an emanation amount of acetone is B1, an emanation amount of acetic acid is B2, and an emanation amount of acetaldehyde is B3, a metabolic index value ME can be calculated by the following formula (2).

$$ME = B 1 / ( B 1 + B 2 + B 3 ) \qquad ( 2 )$$

That is, the metabolic index value ME may be a ratio of the emanation amount of acetone to the total emanation amount of acetone, acetic acid, and acetaldehyde. Note that the above-described method for calculating the metabolic index value ME is an example, and the selection of a plurality of types of metabolism-related gases and the selection of a specific metabolism-related gas are not limited to the above. For example, the metabolic index value ME may be a ratio of the emanation amount of the total emanation amount of acetic acid and acetaldehyde to the total emanation amount of acetone, acetic acid, and acetaldehyde (ME′=(B2+B3)/(B1+B2+B3)).

As described above, by using the metabolic index value ME, which is the ratio of the emanation amount of the specific metabolism-related gas to the total emanation amount of the plurality of types of metabolism-related gases, it is possible to suppress the influence of the variation in the emanation amount of the skin gas caused by climatic conditions or factors unique to the subject (physical constitution, mental state, and the like).

The determination unit 104 determines the stress state of the subject on the basis of at least one index value. Specifically, based on the blood vessel index value BV calculated by the blood vessel index value calculation unit 102 and the metabolic index value ME calculated by the metabolic index value calculation unit 103, the determination unit 104 determines whether or not the subject is in the stress state in which the subject feels stress of a predetermined degree or more.

Note that the blood vessel index value BV and the metabolic index value ME, which are indices used for determination by the determination unit 104, are index values calculated on the basis of chemical component analysis, and thus can be referred to as objective index values. In addition, the blood vessel index value BV and the metabolic index value ME are examples of objective index values. The determination unit 104 may determine whether or not the subject is in the stress state on the basis of index values other than the blood vessel index value BV and the metabolic index value ME.

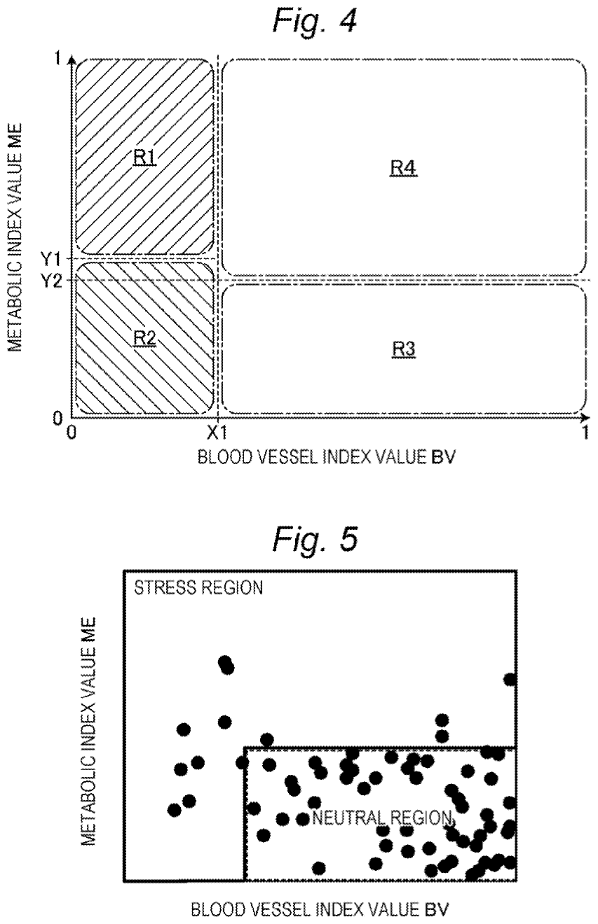

FIG. 4 is a diagram illustrating an example of a stress state map showing a relationship between the blood vessel index value BV, the metabolic index value ME, and the stress state. An example of a determination method by the determination unit 104 will be described with reference to the stress state map illustrated in FIG. 4.

The determination unit 104 determines that the subject is in the stress state when the relationship between the blood vessel index value BV and a threshold value X1 satisfies a predetermined condition. For example, in a case where the blood vessel index value BV is a ratio of hexanoic acid to the total emanation amount of hexanoic acid and propionic acid, when the blood vessel index value BV is equal to or less than the threshold value X1, that is, when a point (BV, ME) is within a region R1 or a region R2 in the graph illustrated in FIG. 4, it is determined that the subject is in the stress state. Note that the threshold value X1 and the predetermined condition (such as a magnitude relationship between BV and X1) change according to the selection of the blood-vessel-related gas and the specific blood-vessel-related gas.

When it is determined that the subject is in the stress state, the determination unit 104 determines whether the subject is in a physical stress state in which the subject feels physical stress or a mental stress state in which the subject feels mental stress on the basis of the relationship between the metabolic index value ME and a threshold value Y1. For example, in a case where the metabolic index value ME is the ratio of the emanation amount of acetone to the total emanation amount of acetone, acetic acid, and acetaldehyde, when the blood vessel index value BV is equal to or less than the threshold value X1 and the metabolic index value ME is equal to or greater than the threshold value Y1, that is, when the point (BV, ME) is within the region R1 in the graph illustrated in FIG. 4, it is determined that the subject is in the physical stress state.

Further, when the blood vessel index value BV is equal to or less than the threshold value X1 and the metabolic index value ME is less than the threshold value Y1, that is, when the point (BV, ME) is within the region R2 in the graph illustrated in FIG. 4, the determination unit 104 determines that the subject is in the mental stress state. The mental stress refers to a stress response caused by a stress stimulus (stressor) in a psychological aspect, and refers to, for example, stress accompanied by symptoms such as decline in energy, irritation, anxiety, and depression (decline in mood, decrease in interest and concern).

Further, when the blood vessel index value BV is greater than the threshold value X1 and the metabolic index value ME is equal to or less than a threshold value Y2, that is, when the point (BV, ME) is within a region R3 in the graph illustrated in FIG. 4, the determination unit 104 determines that the subject is in a neutral state. The neutral state is not a stress state but may be a mentally and physically stable state.

Further, when the blood vessel index value BV is greater than the threshold value X1 and the metabolic index value ME is greater than the threshold value Y2, that is, when the point (BV, ME) is within a region R4 in the graph illustrated in FIG. 4, the determination unit 104 determines that the subject is in a temporary high activity state. The temporary high activity state is not a stress state corresponding to the region R1 and the region R2, but is a state in which a physical load is higher than usual, which can be also considered as a kind of stress state. Note that the conditions for dividing the threshold values Y1 and Y2 and the regions R1 to R4 (such as the magnitude relationship among ME, Y1, and Y2) change according to the selection of the metabolism-related gas and the specific metabolism-related gas.

According to the determination method as described above, it is possible to determine whether or not the subject is in the stress state on the basis of the blood vessel index value BV. Further, it is possible to determine whether the type of stress felt by the subject is physical or mental on the basis of the metabolic index value ME in addition to the blood vessel index value BV. In addition, it is also possible to determine other states (for example, a neutral state) other than the stress state. Such a determination result regarding other states may be used for generating stress information and support information, for example.

In FIG. 4, a case where the stress state is classified into four states (regions) of a “physical stress state”, a “mental stress state”, a “neutral state”, and a “temporary high activity state” is exemplified. On the other hand, the number of stress state classifications can be arbitrarily set as necessary.

FIG. 5 is a diagram illustrating another example of the stress state map showing the relationship between the blood vessel index value BV, the metabolic index value ME, and the stress state. In the example illustrated in FIG. 5, the stress state is classified into two states of “stress state” and “neutral state”.

Further, for example, as indicated by a plurality of black dots in FIG. 5, it is also possible to grasp the temporal change of the stress state by mapping points (BV, ME) corresponding to skin gases having different acquisition times on the stress state map.

Further, the determination unit 104 executes body odor classification processing on the subject on the basis of the determined stress state. Specifically, the determination unit 104 executes body odor classification of the subject by using the determined stress state and body odor classification information.

Here, the body odor classification information is a table (or database) in which the stress state and the body odor classification are associated with each other. For example, the body odor classification information is a table in which the stress states and the body odor classifications are associated with each other by associating the stress state “physical stress state” with the body odor classification “pungent fermentation odor (rotten cheese odor)”, the stress state “mental stress state” with the body odor classification “pungent sour odor (odor like vinegar)”, the stress state “temporary high activity state” with the body odor classification “rubber odor”, and the stress state “neutral state” with the body odor classification “neutral odor (not characteristic pungent odor)”.

In the present embodiment, the case where the body odor classification is classified into four types of “pungent fermentation odor (rotten cheese odor)”, “pungent sour odor (odor like vinegar)”, “rubber odor”, and “neutral odor (not characteristic pungent odor)” has been exemplified. On the other hand, the number of body odor classifications can be arbitrarily set as necessary. The number of body odor classifications is not necessarily the same as the number of stress state classifications, and the body odor classification information may be any information as long as a plurality of stress states and a plurality of body odor classifications are associated with each other.

The generation unit 105 generates body odor care support information regarding the subject on the basis of the body odor classification by the determination unit 104.

Here, the body odor care support information is information including at least one of a result of the body odor classification of the subject and information regarding body odor suppression based on the body odor classification. Further, the information regarding body odor suppression is information including at least one of a treatment (radical treatment) for reducing in-vivo generation of a substance that causes body odor in the body odor classification and a treatment (countermeasure treatment) for suppressing (generated) body odor in the body odor classification. Further, the body odor care support information may include information for reducing stress of the subject.

For example, in a case where the body odor classification is “pungent fermentation odor (rotten cheese odor)”, the body odor care support information is information such as “aerobic exercise”, “dietary life review (refraining from taking . . . )”, and “sufficient sleep”. In addition, in a case where the body odor classification is “pungent sour odor (odor like vinegar)”, the body odor care support information is information such as “exercise, bathing (sweat gland training)”. Further, in a case where the body odor classification is “rubber odor”, the body odor care support information is information such as “refraining from excessive drinking and eating”, “refraining from drinking alcohol”, and “refraining from eating and drinking late at night”.

Note that various methods can be adopted as a method by which the generation unit 105 generates the body odor care support information. For example, the generation unit 105 can generate the body odor care support information using a database (or table) in which a plurality of body odor classifications and a plurality of pieces of information regarding body odor suppression are associated with each other. Further, the generation unit 105 can also generate the body odor care support information by using, for example, a learned model that inputs a body odor classification and outputs body odor care support information such as the plurality of pieces of information regarding body odor suppression.

The output unit 106 outputs (presents) the body odor care support information generated by the generation unit 105 in a predetermined format.

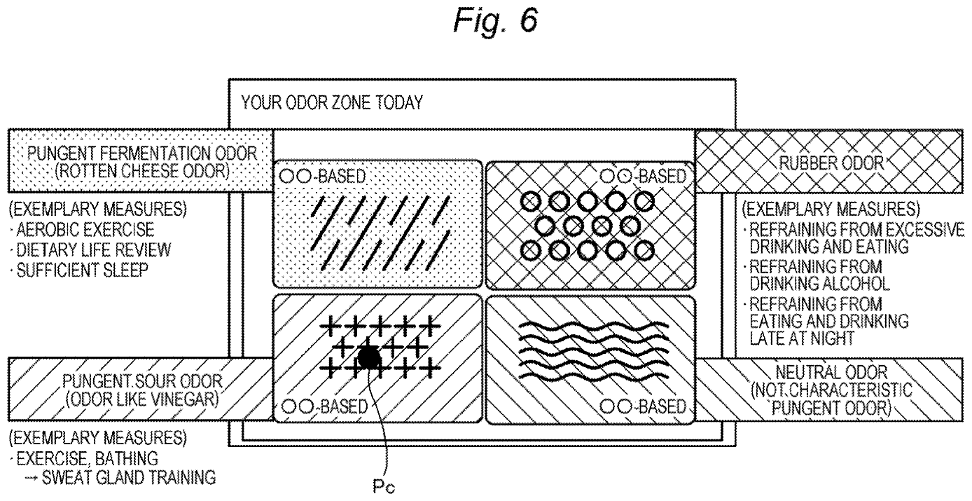

FIG. 6 is a diagram illustrating an example of a form of presenting the body odor care support information. In the example illustrated in FIG. 6, a point Pc on the simplified stress state map indicates which region (zone) the subject is classified (that is, the body odor classification of the subject). Further, a plurality of pieces of information regarding body odor suppression for each body odor classification is also presented at the same time.

With the body odor care support information presented as illustrated in FIG. 6, the subject can easily and quickly acquire and confirm his/her body odor classification and the support information for body odor care according to the body odor classification.

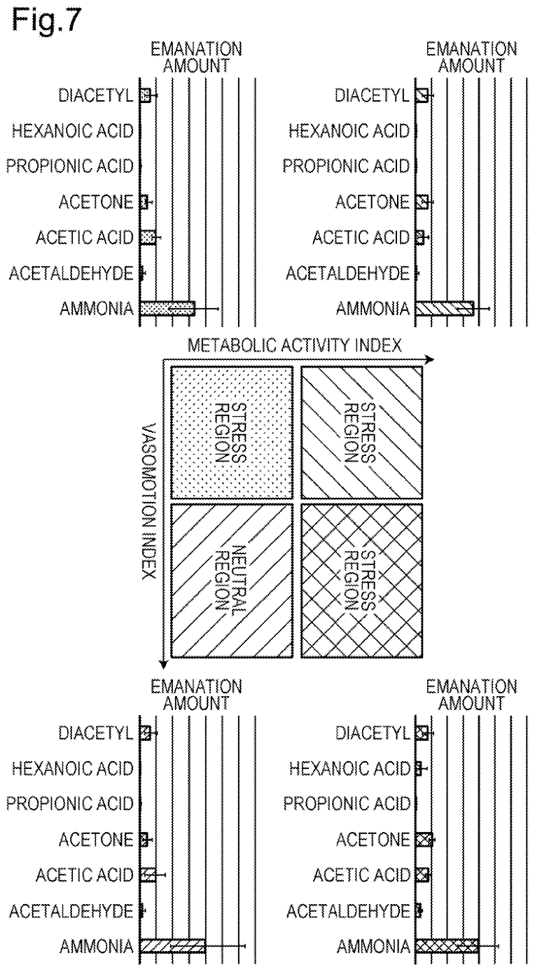

FIG. 7 is a diagram illustrating another example of a form of presenting the body odor care support information. In the example illustrated in FIG. 7, in addition to the simplified stress state map, a typical skin gas component (and a skin gas component of the subject) for each body odor classification is also presented at the same time.

With the body odor care support information presented as illustrated in FIG. 7, the subject can easily and quickly acquire and confirm his/her body odor classification, a dominant component for the body odor care of the subject, and the like.

The storage unit 107 stores the skin gas information acquired by the acquisition unit 101, the blood vessel index value BV calculated by the blood vessel index value calculation unit 102, the metabolic index value ME, the determination result by the determination unit 104, the body odor care support information generated by the generation unit 105, and the like for each subject.

### Body Odor Care Support Information Generation Processing

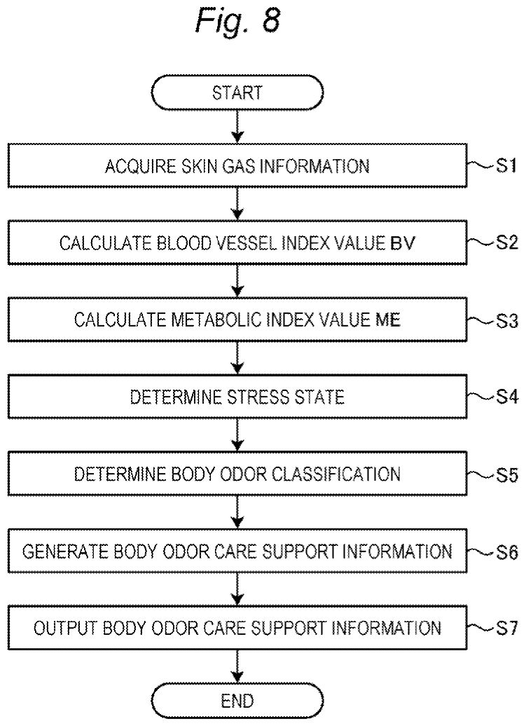

FIG. 8 is a flowchart illustrating an example of a flow of body odor care support information generation processing according to the embodiment. The body odor care support information generation processing will be described with reference to FIG. 8.

First, the acquisition unit 101 acquires skin gas information from the skin gas sensor 11 (step S1).

The blood vessel index value calculation unit 102 calculates the blood vessel index value BV on the basis of the skin gas information acquired by the acquisition unit 101 (step S2). Note that the blood vessel index value BV is a ratio of the emanation amount of hexanoic acid to the total emanation amount of hexanoic acid and propionic acid.

The metabolic index value calculation unit 103 calculates the metabolic index value ME on the basis of the skin gas information acquired by the acquisition unit 101 (step S3). Note that the metabolic index value ME is a ratio of the emanation amount of acetone to the total emanation amount of acetone, acetic acid, and acetaldehyde.

The determination unit 104 determines the stress state of the subject using the calculated blood vessel index value BV and metabolic index value ME, and the stress state map (step S4).

Further, the determination unit 104 executes the body odor classification processing on the subject on the basis of the determined stress state, and determines the body odor classification of the subject (step S5).

The generation unit 105 generates body odor care support information regarding the subject on the basis of the body odor classification by the determination unit 104 (step S6).

The output unit 106 outputs the generated stress information in a predetermined format (step S7).

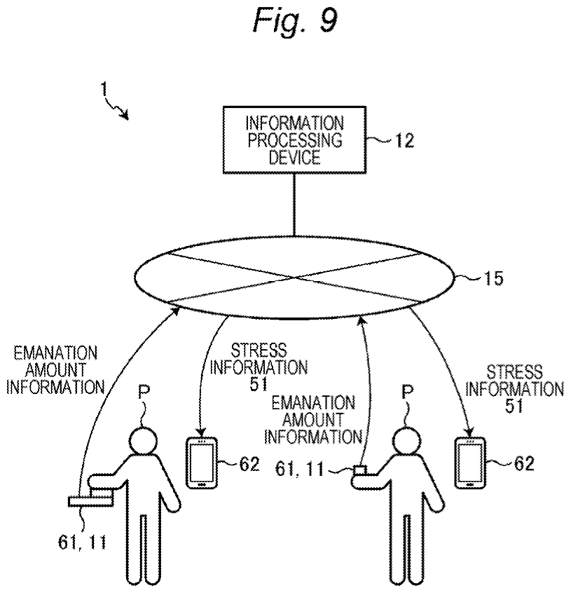

FIG. 9 is a diagram illustrating an example of a form of providing the body odor care support information to the subject. In the body odor care support information providing system 1 exemplified in FIG. 9, the body odor care support information providing device 12 acquires the emanation amount information corresponding to each of a plurality of subjects P from a predetermined detection device 61 via the network 15, generates body odor care support information 51 of each subject P on the basis of each piece of acquired emanation amount information, and respectively transmits pieces of generated body odor care support information 51 to terminal devices 62 respectively used by the corresponding subjects P via the network 15.

The form of the detection device 61 (the device on which the skin gas sensor 11 is mounted) is not particularly limited, and may be, for example, a dedicated device fixedly installed at a specific place, a wearable device that can be worn by the subject P, or the like. The form of the terminal device 62 used by the subject P should not be particularly limited, and may be, for example, a smartphone, a tablet terminal, a personal computer, a dedicated device specialized for use in the body odor care support information providing system 1, or the like.

As described above, the body odor care support information providing device 12 according to the present embodiment includes the acquisition unit 101, the blood vessel index value calculation unit 102, the metabolic index value calculation unit 103, the determination unit 104, the generation unit 105, and the output unit 106. The acquisition unit 101 acquires the emanation amount information regarding emanation amounts of a plurality of types of skin gases emanated from the skin of the subject. Based on the emanation amount information, the blood vessel index value calculation unit 102 and the metabolic index value calculation unit 103 calculate the blood vessel index value BV and the metabolic index value ME each indicating a ratio of an emanation amount of a specific skin gas selected from a plurality of types of skin gases. The determination unit 104 determines, based on the calculated blood vessel index value BV and metabolic index value ME, the stress state of the subject, and executes, based on the determined stress state, the body odor classification of the subject. The generation unit 105 generates, based on the body odor classification of the subject, body odor care support information regarding the subject. The output unit 106 outputs the body odor care support information.

Therefore, according to the body odor care support information providing device 12, the stress state can be correctly determined based on the objective index value calculated using the skin gas of the subject, and the highly objective body odor classification can be executed based on the determined stress state. Further, specific and appropriate body odor care support information for the subject can be presented on the basis of the highly objective body odor classification. The subject can quickly and easily acquire the body odor care support information only by providing his/her skin gas, and can realize effective body odor care using the body odor care support information.

Further, the body odor care support information includes at least one of a result of the body odor classification of the subject and information regarding body odor suppression based on the body odor classification.

Therefore, according to the body odor care support information providing device 12, the subject can grasp, from the result of the body odor classification of the subject, what classification his/her body odor specifically belongs to, such as “pungent fermentation odor (rotten cheese odor)”, “pungent sour odor (odor like vinegar)”, “rubber odor”, and “neutral odor (not characteristic pungent odor)”. Further, according to the information regarding body odor suppression based on the body odor classification, it is possible to grasp a treatment (radical treatment) for reducing in-vivo generation of a substance that causes body odor in the body odor classification and a treatment (countermeasure treatment) for suppressing (generated) body odor in the body odor classification and to take measures and treatments.

Further, the at least one index value includes at least one of the blood vessel index value and the metabolic index value.

Therefore, according to the body odor care support information providing device 12, it is possible to acquire the body odor care support information and take specific measures against the body odor caused by at least one of the physical stress state and the mental stress state.

### First Modification

In the above embodiment, the case where the determination of the stress state is executed using the two indices of the blood vessel index value BV and the metabolic index value ME and the stress state map has been taken as an example. On the other hand, for example, it is also possible to execute determination of the stress state using one of the blood vessel index value BV and the metabolic index value ME.

FIG. 10 is a diagram illustrating an example of a relationship between the blood vessel index value BV and a stress value S. In FIG. 10, a line L1 indicating the relationship between the blood vessel index value BV and the stress value S is exemplified. The stress value S is obtained by quantifying the level of the mental stress on the basis of a predetermined standard, and is set to be a higher value as the stress is stronger.

FIG. 10 shows that there is a relationship in which the blood vessel index value BV decreases as the stress value S increases. Here, in a case where it is set that the subject is in the stress state when the stress value S is equal to or greater than a threshold value S1, it can be determined that the subject is in the stress state when the blood vessel index value BV is equal to or less than the threshold value X1 corresponding to the threshold value S1.

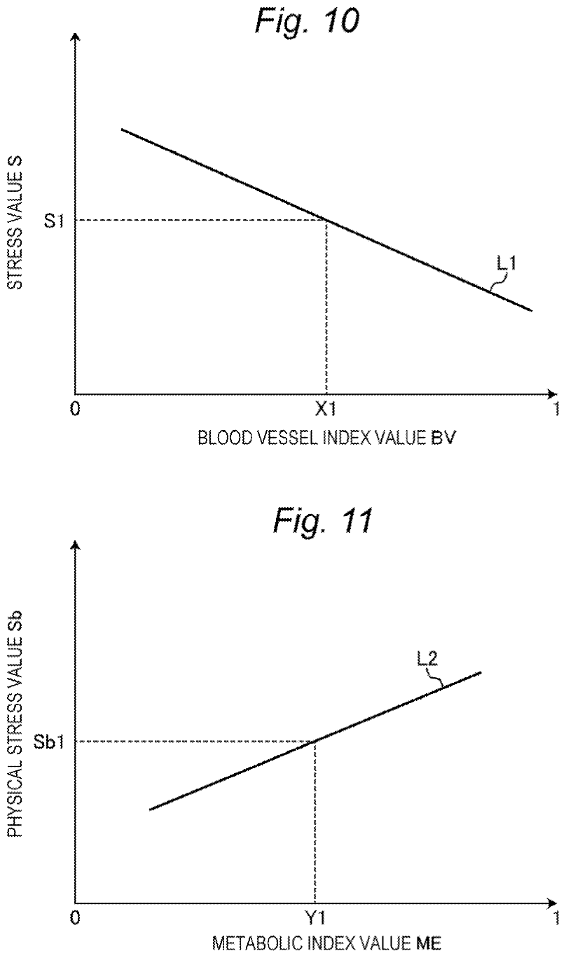

FIG. 11 is a diagram illustrating an example of a relationship between the metabolic index value ME and a physical stress value Sb. In FIG. 11, a line L2 indicating the relationship between the metabolic index value ME and the physical stress value Sb is exemplified. The physical stress value Sb is obtained by quantifying the level of the physical stress on the basis of a predetermined standard, and is set to be a higher value as the physical stress is stronger.

FIG. 11 shows that there is a relationship in which the metabolic index value ME increases as the physical stress value Sb increases. Here, in a case where it is set that the subject is in the physical stress state when the physical stress value Sb is equal to or greater than a threshold value Sb1, it can be determined that the subject is in the physical stress state when the metabolic index value ME is equal to or greater than the threshold value Y1 corresponding to the threshold value Sb1.

The determination unit 104 can also determine the stress state of the subject on the basis of the relationship in, for example, FIG. 10 or 11, and execute the body odor classification processing of the subject based on the determined stress state.

According to the body odor care support information providing device 12 of the modification, determination of the stress state is executed using one of the blood vessel index value BV and the metabolic index value ME. Therefore, it is possible to reduce the calculation load and simplify the device as compared with the case of using both the blood vessel index value BV and the metabolic index value ME.

### Second Modification

In the above embodiment, the case where the object to be measured for body odor is a subject (human) is taken as an example. On the other hand, the object to be measured for body odor can also be animals other than humans (pets, livestock, animals raised in zoos).

A program for causing a computer to execute processing for realizing a calculation function and a control function of the body odor care support information providing device described above can be provided by being recorded in a computer-readable recording medium such as a compact disc (CD)-ROM, a flexible disk (FD), a CD-recordable (R), and a digital versatile disk (DVD) as a file in an installable format or an executable format. Further, the program may be provided or distributed via a network such as the Internet.

Although the embodiment of the present disclosure has been described above, the above-described embodiment and modifications thereof are merely examples, and are not intended to limit the scope of the disclosure. The above-described novel embodiment and modifications can be implemented in various forms, and various omissions, substitutions, and changes can be made without departing from the gist of the disclosure. The above-described embodiment and modifications are included in the scope and gist of the disclosure, and are included in the disclosure described in the claims and the equivalent scope thereof.

### Summary of Present Embodiment

The present embodiment includes at least the following configuration.

A body odor care support information providing device (12) according to the embodiment includes: an acquisition unit (101) that acquires emanation amount information regarding emanation amounts of a plurality of types of skin gases emanated from a skin of a subject; a calculation unit (102, 103) that calculates, based on the emanation amount information, at least one index value indicating a ratio of an emanation amount of a specific skin gas selected from the plurality of types of skin gases; a determination unit (104) that determines, based on the at least one index value, a stress state of the subject, and determines, based on the determined stress state, a body odor classification of the subject; a generation unit (105) that generates, based on the body odor classification of the subject, body odor care support information regarding the subject; and an output unit (106) that outputs the body odor care support information.

According to such a configuration, the stress state can be correctly determined based on the objective index value calculated using the skin gas of the subject, and the highly objective body odor classification can be executed based on the determined stress state. Further, specific and appropriate body odor care support information for the subject can be presented on the basis of the highly objective body odor classification. The subject can quickly and easily acquire the body odor care support information only by providing his/her skin gas, and can realize effective body odor care using the body odor care support information.

Further, the body odor care support information includes at least one of a result of the body odor classification of the subject and information regarding body odor suppression based on the body odor classification.

Therefore, according to the body odor care support information providing device 12, the subject can grasp, from the result of the body odor classification of the subject, what classification his/her body odor specifically belongs to, such as “pungent fermentation odor (rotten cheese odor)”, “pungent sour odor (odor like vinegar)”, “rubber odor”, and “neutral odor (not characteristic pungent odor)”. Further, according to the information regarding body odor suppression based on the body odor classification, it is possible to grasp a treatment (radical treatment) for reducing in-vivo generation of a substance that causes body odor in the body odor classification and a treatment (countermeasure treatment) for suppressing (generated) body odor in the body odor classification and to take measures and treatments.

Further, the at least one index value includes at least one of the blood vessel index value and the metabolic index value.

Therefore, according to the body odor care support information providing device 12, it is possible to acquire the body odor care support information and take specific measures against the body odor caused by at least one of the physical stress state and the mental stress state.

A body odor care support information providing method using the body odor care support information providing device (12) according to the embodiment includes: acquiring emanation amount information regarding emanation amounts of a plurality of types of skin gases emanated from a skin of a subject; calculating, based on the emanation amount information, at least one index value indicating a ratio of an emanation amount of a specific skin gas selected from the plurality of types of skin gases; determining, based on the at least one index value, a stress state of the subject, determining, based on the determined stress state, a body odor classification of the subject; generating, based on the body odor classification of the subject, body odor care support information regarding the subject; and outputting the body odor care support information.

According to such a configuration, the stress state can be correctly determined based on the objective index value calculated using the skin gas of the subject, and the highly objective body odor classification can be executed based on the determined stress state. Further, specific and appropriate body odor care support information for the subject can be presented on the basis of the highly objective body odor classification. The subject can quickly and easily acquire the body odor care support information only by providing his/her skin gas, and can realize effective body odor care using the body odor care support information.

Although the embodiment of the present disclosure has been exemplified above, the above-described embodiment and modifications are merely examples, and are not intended to limit the scope of the disclosure. The above-described embodiment and modifications can be implemented in various other forms, and various omissions, substitutions, combinations, and changes can be made without departing from the gist of the disclosure. In addition, the configuration and shape of each embodiment and each modification can be implemented by being partially interchanged.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

1. A body odor care support information providing device comprising:
an acquisition unit that acquires emanation amount information regarding emanation amounts of a plurality of types of skin gases emanated from a skin of a subject;
a calculation unit that calculates, based on the emanation amount information, at least one index value indicating a ratio of an emanation amount of a specific skin gas selected from the plurality of types of skin gases;
a determination unit that determines, based on the at least one index value, a stress state of the subject, and determines, based on the determined stress state, a body odor classification of the subject;
a generation unit that generates, based on the body odor classification of the subject, body odor care support information regarding the subject; and
an output unit that outputs the body odor care support information.

2. The body odor care support information providing device according to claim 1,
wherein the body odor care support information includes at least one of a result of the body odor classification of the subject and information regarding body odor suppression based on the body odor classification.

3. The body odor care support information providing device according to claim 1,
wherein the at least one index value includes at least one of a blood vessel index value and a metabolic index value.

4. The body odor care support information providing device according to claim 2,
wherein the at least one index value includes at least one of a blood vessel index value and a metabolic index value.

5. A body odor care support information providing method using a body odor care support information providing device, the body odor care support information providing method comprising:
acquiring emanation amount information regarding emanation amounts of a plurality of types of skin gases emanated from a skin of a subject;
calculating, based on the emanation amount information, at least one index value indicating a ratio of an emanation amount of a specific skin gas selected from the plurality of types of skin gases;
determining, based on the at least one index value, a stress state of the subject,
determining, based on the determined stress state, a body odor classification of the subject;
generating, based on the body odor classification of the subject, body odor care support information regarding the subject; and
outputting the body odor care support information.
